(19) United States
(12) Patent Application Publication
(10) Pub. No.: US 2003/0162707 A1
(43) Pub. Date: Aug. 28, 2003

(54) Systems and methods for treating patients with collagen-rich material extracted from adipose tissue

(76) Inventors: John K. Fraser, Los Angeles, CA (US); Marc H. Hedrick, Encino, CA (US); Ramon Llull, Mallorca (ES)

Correspondence Address: Stout, Uxa, Buyan & Mullins, LLP, Suite 300, 4 Venture, Irvine, CA, 92618, US

(21) Appl. No.: 10/325,728

(22) Filed: Dec. 20, 2002

Related U.S. Application Data
(60) Provisional application No. 60/342,910, filed on Dec. 20, 2001.

(51) Int. Cl. A61K 38/17

(52) U.S. Cl. 514/12

(57) ABSTRACT

Compositions, methods, and systems are disclosed for using collagen-rich material, derived from adipose tissue, that is placed directly into a recipient along with such additives necessary to promote, engender, or support a therapeutic, structural, or cosmetic benefit. The compositions may be obtained during the course of a single surgical procedure, and may be administered to the patient immediately after adipose tissue is removed from a patient, such as within hours or days from being withdrawn from the patient.

### CROSS-REFERENCE TO RELATED APPLICATIONS

This application claims the benefit of U.S. Provisional Application No. 60/342,910, entitled EXTRACTION, STORAGE, AND APPLICATION OF ADIPOSE TISSUE-DERIVED COLLAGEN-CONTAINING MATERIAL, filed Dec. 20, 2001, the entire contents of which are hereby incorporated by reference.

### BACKGROUND OF THE INVENTION

1. Field of the Invention

This invention generally relates to connective tissue material derived from adipose tissue, and more particularly, to adipo-derived collagen-rich material, methods of using adipo-derived collagen-rich material, compositions containing adipo-derived collagen-rich material, and systems for preparing and using adipo-derived collagen-rich material.

b 2. Description of Related Art

Collagen is one of the basic structural proteins of the human body (Bergeon 1967). It provides the core framework of bone, connective soft tissues, and skin (Uitto 1971; Liu, Yang et al. 1995). Collagen is increasingly used in medical devices, especially in the area of soft tissue repair and augmentation (Kamer and Churukian 1984; Klein 2001; Sclafani and Romo 2001). This includes use in hemostatic sponges (Purna and Babu 2000), in drug delivery, as a matrix for cell-based products (Silver and Pins 1992; Scherberich and Beretz 2000), skin repair, vocal cord repair (Ford, Staskowski et al. 1995; Remacle, Lawson et al. 1999), ophthalmic application (Hamel, Shaarawy et al. 2001), and in soft tissue augmentation (wrinkle filler or other support functions) (Kamer and Churukian 1984). The majority of collagen used for such applications has traditionally been of bovine origin (Aragona, D'Urso et al. 1998) although more recently recombinant collagen is being developed (Bulleid, John et al. 2000; Myllyharju 2000). Both these sources have potential problems; with collagen of bovine origin these are largely the presence of adventitious agents (Aragona, D'Urso et al. 1998) and induction of an immune response while recombinant collagen remains an unproven entity.

Native human collagen and collagen-rich material extracted from human skin (Sclafani, Romo et al. 2002) and fascia (Shore 2000; Burres 2001), have also been used in cosmetic applications although these tissue sources are not readily amenable to “real time” processing into a usable product at the patient's bedside and current commercialization has been restricted to applications in which tissue from one individual, almost always cadaveric, is prepared for injection into a recipient individual. This approach is subject to risks of introduction of adventitious organisms, for example hepatitis viruses or HIV. The FDA literature contains at least one case of product recall of a product for this reason (Jan. 16, 2001 Alloderm; LifeCell Inc.)—cited on FDA Website.

This use of bovine or porcine collagen is subject to problems due to allergic responses (Boerner 1988; Mullins, Richards et al. 1996) and rapid immune system-mediated degradation of the implant (Aragona, D'Urso et al. 1998) resulting in limited durability of response in cosmetic and therapeutic applications (Groutz, Blaivas et al. 2000; Yokoyama, Yoshimura et al. 2001; Block, Cooper et al. 2003).

These shortcomings could be ameliorated by use of an autologous (self-derived) product with the same properties as xenogeneic (derived from another species) or allogeneic (derived from another individual of the same species) collagen. Some investigators have attempted to use whole or fragmented adipose tissue as a source of soft tissue filler material for many of the applications for which bovine collagen is commonly applied (Boering and Huffstadt 1967; Asken 1990; Koufman 1991; Coleman, Lawrence et al. 1993; Carpaneda 1994; Haab, Zimmern et al. 1997; Hsiung, Woo et al. 2000; Coleman 2001; Lee, Kung et al. 2001). However in these applications there can be problems associated with inflammatory and fibrotic responses to the implanted fat which frequently necroses due to lack of vascularity and to the occasional opposite response where the fat undergoes hypertrophy and a problem-causing overcorrection is observed (Bartynski, Marion et al. 1990; Nguyen, Pasyk et al. 1990; Latoni, Marshall et al. 2000; Miller and Popp 2002).

There remains a need in the art for new methods and devices for processing adipose tissue to inexpensively and reliably provide high yields of collagen-rich material that does not induce significant, if any, inflammatory and/or immune responses when administered to patients.

### SUMMARY OF THE INVENTION

The present invention is directed to compositions, methods, and systems for using collagen-rich material derived from adipose tissue that is placed directly into a recipient along with such additives necessary to promote, engender, or support a therapeutic, structural, or cosmetic benefit. The compositions may be obtained during the course of a single surgical procedure, and may be administered to a patient immediately after adipose tissue is removed from a patient, such as within hours or days from being withdrawn from the patient.

In one embodiment, adipose tissue processing occurs in a system that maintains a closed, sterile fluid/tissue pathway. This is achieved by use of a pre-assembled, linked set of closed, sterile containers and tubing allowing for transfer of tissue and fluid elements within a closed pathway. This processing set can be linked to a series of processing reagents (e.g., saline, detergents, etc.) inserted into a device which can control the addition of reagents, temperature, and timing of processing thus relieving operators of the need to manually manage the process. In a preferred embodiment the entire procedure from tissue extraction through processing and placement into the recipient would all be performed in the same facility, indeed, even within the same room of the patient undergoing the procedure.

In accordance with one aspect of the invention, raw adipose tissue is processed to substantially remove and the cellular components thereby obtaining a heterogeneous connective tissue matrix material that is rich in collagen that is suitable for placement within the body of a recipient. The collagen-rich material may be placed into the recipient in combination with cells, tissue, tissue fragments, or other stimulators of cell growth and/or differentiation. In a preferred embodiment, the material, with any of the above mentioned additives, is placed into the person from whom they were obtained in the context of a single operative procedure with the intention of deriving a therapeutic, structural, or cosmetic benefit to the recipient.

In one embodiment, a method of treating a patient includes steps of: a) providing a tissue removal system; b) removing adipose tissue from a patient using the tissue removal system, the adipose tissue having a concentration of collagen-rich material; c) processing at least a part of the adipose tissue to obtain a concentration of collagen other than the concentration of collagen of the adipose tissue before processing; and d) administering the collagen-rich material to a patient without removing the collagen-rich material from the tissue removal system before being administered to the patient.

A system in accordance with the invention herein disclosed includes a) a tissue collection container including i) a tissue collecting inlet port structured to receive adipose tissue removed from a patient; and ii) a filter disposed within the container and being structured to retain adipose tissue removed from a patient and to pass non-adipose tissue removed from the patient; b) a mixing container coupled to the tissue collection container to receive collagen-rich material obtained from the adipose tissue without removal of the collagen-rich material from the tissue removal system, and including an additive port for the administration of at least one additive to mix with the collagen-rich material contained therein; and c) an outlet structured to permit the collagen-rich material in the mixing container to be removed from the tissue collection system for administration to a patient.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description.

### BRIEF DESCRIPTION OF THE DRAWINGS

FIG. 1 depicts a tissue removal system for processing adipose tissue to extract collagen-rich material from the adipose tissue; and

FIG. 2 depicts a processing device for automating the operation of a tissue removal system as illustrated in FIG. 1.

### DETAILED DESCRIPTION OF THE PRESENTLY PREFERRED EMBODIMENTS

Reference will now be made in detail to the presently preferred embodiments of the invention, examples of which are illustrated in the accompanying drawings. Wherever possible, the same or similar reference numbers are used in the drawings and the description to refer to the same or like parts. It should be noted that the drawings are in simplified form and are not to precise scale. In reference to the disclosure herein, for purposes of convenience and clarity only, directional terms, such as, top, bottom, left, right, up, down, over, above, below, beneath, rear, and front, are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent of the following detailed description, although discussing exemplary embodiments, is to be construed to cover all modifications, alternatives, and equivalents of the embodiments as may fall within the spirit and scope of the invention as defined by the appended claims. The present invention may be practiced in conjunction with various cell or tissue separation techniques that are conventionally used in the art, and only so much of the commonly practiced process steps are included herein as are necessary to provide an understanding of the present invention.

The present invention is directed to a collagen-rich material present in adipose tissue, and systems and methods for administering the collagen-rich material into a human or animal patient. The collagen-rich material of the adipose tissue may be used as a source of material for therapeutic and cosmetic applications. Among other things, the material may be used for regenerative medicine, such as diseases that can be treated with regenerating cells in which the collagen-rich material acts as a substrate or scaffold for the regenerating or newly generating tissue or cell. The collagen-rich material may be administered to a patient with a cellular additive or additional structural components such as artificial (plastic, metal or other compound) implants or supports, additional or other connective tissue, or the collagen-rich material may be administered mixed together with other tissues, as discussed herein. The collagen-rich material disclosed herein is preferably administered to a patient from which the material was obtained.

It has been found that adipose tissue is a rich source of collagen-rich matrix material. This finding may be due, at least in part, to the ease of removal of the major cellular component of adipose tissue, the adipocyte. Thus, in both human and animal studies, processed acellular lipoaspirate (a type of collagen-rich material) is a non-water soluble collagen-rich connective tissue matrix material that comprises at least 2% and more typically more than 5% of the dry weight of the unprocessed adipose tissue. In other words, when extracted unprocessed adipose tissue is dried, more than approximately 5% of the weight of the dried tissue is typically collagen-rich connective tissue matrix material.

As used herein, “adipose tissue” refers to a tissue containing multiple cell types including adipocytes, reticular cells, and microvascular cells. Adipose tissue includes stem cells and endothelial precursor cells. Accordingly, adipose tissue refers to fat including the connective tissue that stores the fat.

As used herein, “unit of adipose tissue” refers to a discrete or measurable amount of adipose tissue. A unit of adipose tissue may be measured by determining the weight and/or volume of the unit. In reference to the disclosure herein, a unit of adipose tissue may refer to the entire amount of adipose tissue removed from a patient, or an amount that is less than the entire amount of adipose tissue removed from a patient. Thus, a unit of adipose tissue may be combined with another unit of adipose tissue to form a unit of adipose tissue that has a weight or volume that is the sum of the individual units. Similar definitions of a “unit” apply to terms such as “collagen-rich material” and “processed acellular lipoaspirate” in that the unit is a discrete amount of these materials.

As used herein, “portion” refers to an amount of a material that is less than a whole. A minor portion refers to an amount that is less than 50%, and a major portion refers to an amount greater than 50%. Thus, a unit of adipose tissue that is less than the entire amount of adipose tissue removed from a patient is a portion of the removed adipose tissue.

As used herein, “collagen-rich material” refers to adipose tissue that has been processed using any means other than the initial washing with sterile water to remove at least a portion of the non-collagen component from the adipose tissue. In one aspect, collagen-rich material refers to adipose tissue that has been processed using any means other than the initial washing with sterile water to remove at least a portion of the cellular component from the connective adipose tissue.

As used herein, “processed acellular lipoaspirate” refers to adipose tissue that has been processed using any means, other than the initial washing with sterile water, to remove all or. substantially all of the cellular component (i.e., cells and cell fragments) from the adipose tissue. The processed acellular lipoaspirate can comprise water-insoluble protein, proteoglycan and other connective tissue elements (in pellet or resuspended form) obtained by washing and separating the connective tissue from the adipose tissue. A pellet of processed lipoaspirate may be obtained by centrifuging a suspension of collagen-rich material so that the material aggregates at the bottom of a centrifuge container. The processed acellular lipoaspirate may be further purified and extracted to yield a product which is specifically enriched for one or more of the elements of the processed acellular lipoaspirate (e.g., collagen). Similarly, the processed acellular lipoaspirate may be admixed with other factors, modified by chemical reaction to affix or remove chemical moieties to alter the solubility or other physical or physiologic properties of the processed acellular lipoaspirate, or components thereof.

In practicing the methods disclosed herein, the material that is administered to a patient is obtained from adipose tissue. Adipose tissue can be obtained by any method known to persons skilled in the art. For example, adipose tissue may be removed from a patient by suction-assisted lipoplasty, ultrasound-assisted lipoplasty, and excisional lipectomy. In addition, the procedures may include a combination of such procedures, such as a combination of excisional lipectomy and suction-assisted lipoplasty. As the tissue or some fraction thereof is intended for reimplantation into a patient, the adipose tissue should be collected in a manner that preserves the integrity of the viability of the connective tissue component and that minimizes the likelihood of contamination of the tissue with potentially infectious organisms, such as bacteria and/or viruses. Thus, the tissue extraction should be performed in a sterile or aseptic manner to minimize contamination. Suction assisted lipoplasty may be desirable to remove the adipose tissue from a patient as it provides a minimally invasive method of collecting tissue with minimal potential for connective tissue damage that may be associated with other techniques, such as ultrasound assisted lipoplasty.

For suction-assisted lipoplastic procedures, adipose tissue is collected by insertion of a cannula into or near an adipose tissue depot present in the patient followed by aspiration of the adipose into a suction device. In one embodiment, a small cannula may be coupled to a syringe, and the adipose tissue may be aspirated using manual force. Using a syringe or other similar device may be desirable to remove relatively moderate amounts of adipose tissue (e.g., from 0.1 ml to several hundred milliliters of adipose tissue) from a patient. Procedures employing these relatively small devices have the advantage that the procedures can be performed with only local anesthesia, as opposed to general anesthesia. Larger volumes of adipose tissue above this range (e.g., greater than several hundred milliliters) may require general anesthesia at the discretion of the donor and the person performing the collection procedure. When larger volumes of adipose tissue are desired to be removed, relatively larger cannulas and automated suction devices may be employed in the procedure.

Excisional lipectomy procedures include, and are not limited to, procedures in which adipose tissue-containing tissues (e.g., skin) is removed as an incidental part of the procedure; that is, where the primary purpose of the surgery is the removal of tissue (e.g., skin in bariatric or cosmetic surgery) and in which adipose tissue is removed along with the tissue of primary interest.

The adipose tissue that is removed from a patient is collected into a device for further processing. As discussed herein, and in one embodiment, the device is designed for and dedicated to the purpose of collecting tissue for manufacture of a processed adipose tissue connective tissue component, which includes collagen. In other embodiments, the device may be any conventional device that is typically used for tissue collection by physicians performing the extraction procedure. Advantageously, the components of the device may be provided as single-use components so that the device is disposable, or in other words, is not capable of being reused for additional procedures on other patients. A disposable device in accordance with an aspect of the invention herein disclosed can obviate a requirement for repeated sterilization of components which may be associated with existing devices, and can reduce a potential of contamination. In addition, the sterility and/or reliability of the device of the invention can be easier to maintain compared to other devices, due, at least in part, to a relatively small number of components associated with the device of the invention. As another result of the relatively small number of components, all or substantially all of which may be disposable, a cost, assembly time, weight and/or physical size of the device can be relatively small; while, on the other hand, a portability and/or reliability of the device can be relatively high. A device in accordance with the invention processes the adipose tissue to obtain one or more units of collagen-rich material by solubilizing specific components of the adipose tissue to permit the solubilized components to be separated from the non-soluble components. This solubilizing of the various components, such as the cellular and lipid components, of the extracted adipose tissue can provide a relatively large yield of collagen.

The amount of tissue collected will be dependent on a number of variables including, but not limited to, the body mass index of the donor, the availability of accessible adipose tissue harvest sites, concomitant and pre-existing medications and conditions (such as anticoagulant therapy), and the clinical purpose for which the tissue is being collected. Experience with bovine collagen suggests that typical implants intended for cosmetic purposes provide approximately 30 mg of collagen per milliliter of implant with the typical procedure involving two to three milliliters of material for a total implant mass of 90 mg. In certain embodiments of the invention, considerably larger harvests are performed such that the patient can receive one injection at the time of harvest with residual material being stored for later application as required or desired. Similarly there will be patients where smaller volume harvests (less than one milliliter but more than 0.1 milliliter may be applied where a smaller amount of collagen is required or where a greater yield of collagen may be achieved.

Patients undergoing treatment in accordance with the disclosure herein can receive a different concentration of collagen-rich material than other treatments employing unprocessed adipose tissue autologous fat grafting (Coleman 2001) Thus, in accordance with an aspect of the invention, the adipose tissue that is removed from a patient can be processed to remove all, substantially all, a majority, or a portion of the cellular components and thereby change the amount or concentration of collagen that is administered to the patient. In one embodiment, the adipose tissue is processed so that the collagen-rich material comprises less than about 4% cells and cell fragments. More preferably, the collagen-rich material is substantially free of cells and cell fragments. In an additional embodiment, the collagen-rich material contains less than approximately 0.1% of cells and cell fragments that were originally present in the tissue. This has the further advantage of attenuating or eliminating any negative consequences of the presence of the cellular components (necrosis and hypertrophy, among other things) (Bartynski, Marion et al. 1990; Latoni, Marshall et al. 2000; Miller and Popp 2002). Indeed, the presence of cells and cell fragments in collagen materials may undesirably cause local inflammatory responses and/or immune responses. By practicing the methods disclosed herein, the likelihood of inflammation and/or immune responses resulting from the administration of the collagen-rich material to a patient can advantageously be reduced. In one particular embodiment, the collagen-rich material comprises a percentage of reticular cellular components, relative to a total amount of cellular components in the collagen-rich material, that is less than or equal to a percentage of reticular cellular components in the removed adipose tissue, relative to a total amount of cellular components present in the removed adipose tissue

In one embodiment of the invention, patients receive a higher concentration of collagen than the concentration of collagen which may typically be present in adipose tissue transplants. This may be due, at least in part, to the types and configurations of tissue collection containers used in the devices and systems of the present invention. A composition of the invention can include a concentration of collagen that is greater than the concentration of collagen found in an equivalent unit of non-processed adipose tissue. In certain embodiments, the composition has a collagen component in which at least 60% of the material (by dry weight) is collagen. Higher concentrations of collagen, such as up to 100%, are also included in other compositions. The collagen-containing composition may be administered with additional components, not originally present in the adipose tissue extracted from the patient, such as cells, cell differentiation factors, growth promoters, immunosuppressive agents, or medical devices, as discussed herein. To obtain certain compositions in which the composition primarily contains collagen, any suitable method for separating the different protein types present in the collagen-rich material (e.g., the processed-acellular lipoaspirate) may be employed, such as the use of differential solubility in high salt, or non-aqueous conditions or combinations thereof (Davis and Mackle 1981; Ooi, Lacy et al. 1991).

Preparation of the collagen-rich material will require depletion of the mature fat-laden adipocyte component of adipose tissue. In addition, the collagen-rich material preferably requires the depletion of the reticular cell component of adipose tissue. This is typically achieved by a series of washing and solubilization steps in which the tissue is first rinsed to reduce the presence of free lipids (released from ruptured adipocytes) and peripheral blood elements (released from blood vessels severed during tissue harvest), and then the cellular components are solubilized by use of hypotonic lysis and/or detergents or high salt washes.

Rinsing is an optional, but preferred, step in which the tissue is mixed with solutions to wash off free lipid and single cell components, such as those components in blood, leaving behind intact adipose tissue fragments. In one embodiment, the adipose tissue that is removed from the patient is mixed with water, isotonic saline or other physiologic solution(s) (e.g., Plasmalyte®, of Baxter Inc or Normosol® of Abbott Labs). Intact adipose tissue fragments can be separated from the free lipid and cells by any means known to persons skilled in the art including, but not limited to, filtration, decantation, sedimentation, or centrifugation techniques. In the illustrated embodiment of the invention, the adipose tissue is separated from non-adipose tissue by employing a filter disposed within a tissue collection container, as discussed herein. In other embodiments, the adipose tissue is separated from non-adipose tissue using a tissue collection container that utilizes decantation, sedimentation, and/or centrifugation techniques to separate the materials.

The intact tissue fragments are then solubilized using conventional techniques or methods, including hypotonic lysis, high salt extraction, use of detergents, such as Tween 20, Triton X-100, or sodium dodecyl sulfate (SDS), organic solvents, enzymatic digestion with single or combinatorial protelolytic enzymes, such as collagenase, trypsin, lipase, liberase H1, as disclosed in U.S. Pat. No. 5,952,215, and pepsin. In modified embodiments, which may be, for example, more expensive, only partially disposable and/or implemented in a non-closed system, the intact tissue fragments can be solubilized using mincing or sheer forces alone or in combination with one or more of the above-mentioned conventional techniques and methods.

For example, as an overview of a method of the invention, the cellular component of the intact tissue fragments may be solubilized by mixing adipose tissue with sterile water (hypotonic lysis), followed by washing with a dilute detergent solution (e.g., 0.1% Tween 20), followed by washing with water to remove substantially all of the detergent. Residual free lipid may then be removed by mixing the material with an organic solvent such as ethanol or acetone. The material is then prepared by removing the organic solvent and preparing the collagen-rich precipitate (processed acellular lipoaspirate) into a form that can be injected into the patient. The amount and concentration of the collagen to be administered to a patient is controlled in by adjusting the amount of tissue that is processed, by adjusting the volume in which the collagen-rich material is resuspended following the final wash, and/or by adjusting the amount of material that is delivered to the patient. For example, and not by way of limitation, in a setting in which a particularly high concentration of collagen is desired, a larger amount of tissue could be processed (for example>50 ml) and, following processing the collagen-rich material, approximately 1 g of the collagen-rich material can be resuspended in a smaller than usual volume (for example 1 ml) yielding a very high collagen concentration (approx. 1 g/ml). This may be performed by fragmenting the collagen-rich material fibrils (i.e., the processed acellular lipoaspirate) by mechanisms including, but not limited to, shear force (e.g., repeated forced passage through small lumen pathways), homogenization by rapidly spinning blades, or sonication. In addition, the collagen-rich material may be resuspended in saline to provide a suspension of collagen-rich material. Thus, the collagen rich material is neither dried nor preserved.

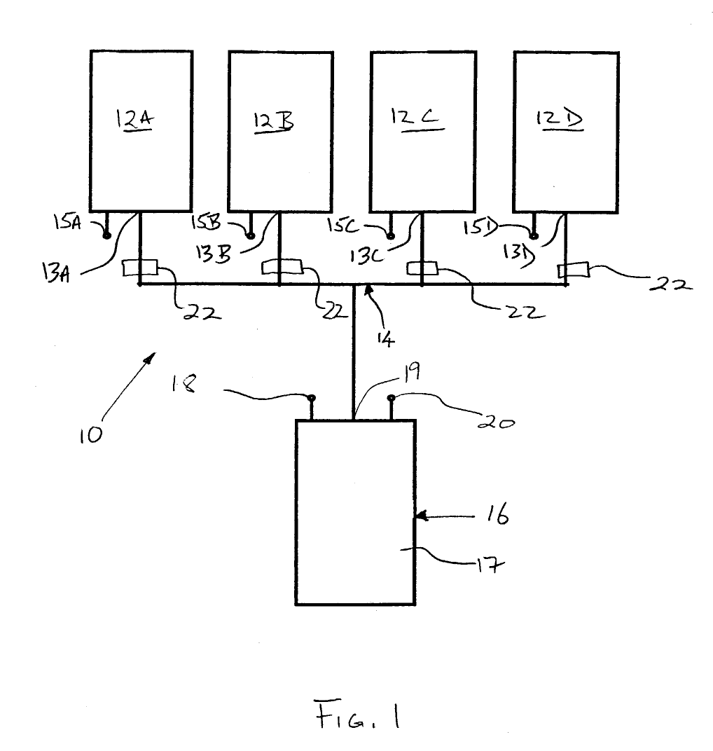

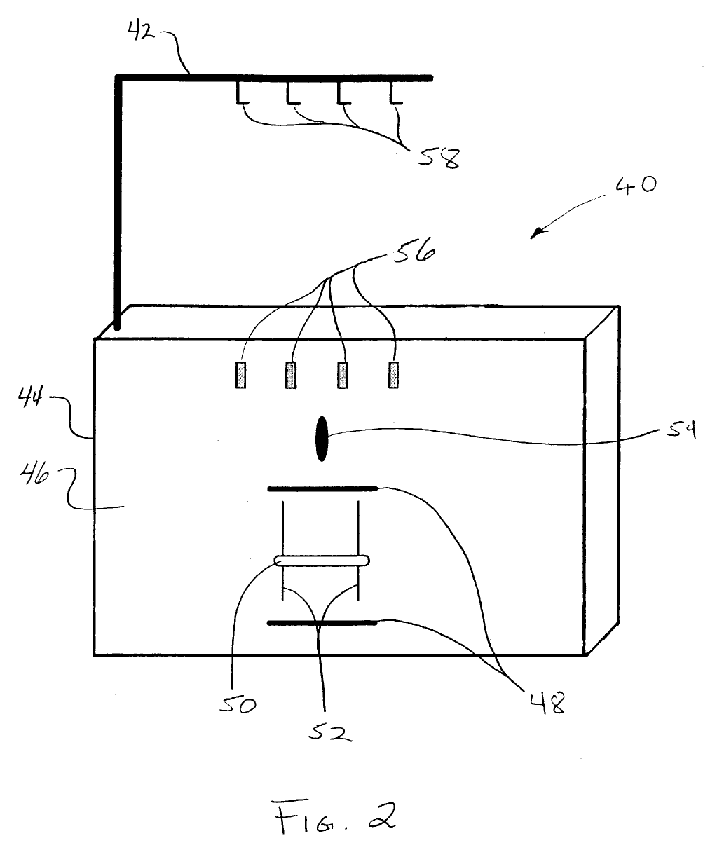

In a preferred embodiment, the processing steps are performed in a single-use disposable device comprised of a set of containers with inlet and outlet ports allowing closed system addition and removal of material from the system. This approach can eliminate the risk of cross-contamination of one specimen with material from another and can reduce the risk of accidental introduction of agents such as bacterial, fungal, or viral pathogens from the environment in which the tissue is processed. An example of a system of this nature is shown in FIG. 1, as discussed in more detail hereinbelow. In another embodiment processing is performed in a device in which the various steps of processing are managed and potentially automated. An example of such a device is shown in FIG. 2, as discussed in more detail hereinbelow.

Further purification of the collagen-rich material may be achieved by solubilization of non-collagen components using high salt buffers, low pH, enzymatic digestion using pepsin, trypsin, papain, or other proteolytic enzymes. Examples of such approaches are described in (Davis and Mackle 1981; Speranza and Valentini 1986; Takasaki, Fujiwara et al. 1995), and U.S. Pat. Nos. 5,436,135, 4,969,912, and 4,597,762. In an exemplary embodiment, the tissue collection container may be a flexible bag that is structured to be placed in a device allowing agitation during the mixing steps of tissue processing (e.g., manually or by robotics). In other embodiments, a flexible bag is not used. After solubilization of the cellular component, the acellular component forms a pellet or mass by removing the solubilized cellular component. The mass may then be resuspended with one or more additional fluids that solubilize various components of the substantially acellular material. The solubilization fluids may be provided by any suitable means. For example, a fluid may be injected into a port on the tissue collection container.

In one exemplary embodiment, between 1 and 50 milliliters of adipose tissue is washed with 50-600 ml sterile buffered isotonic saline and mixed with 100-600 ml of sterile distilled water for 20 minutes at room temperature. The water is then removed by allowing the insoluble component of the collagen-rich material to settle, and directing the soluble component in the water (e.g., the materials contained in the liquid phase of the mixture) from the container, for example, by pressing the container to force the soluble component out, or by withdrawing the soluble component with an aspiration device, such as a syringe. The insoluble component in the container may then be mixed with 100-600 ml of 2.0% Triton X-100 detergent (Sigma Chemical Company, St. Louis, Mo.). Tissue is mixed at room temperature (approximately 20° C.) for 20 minutes. The detergent wash may be repeated as an optional step or steps. The detergent washes enhance the solubilization of the cellular components of the collagen-rich material. The soluble component and detergent mixture may then be removed using a similar method to the removal of the water. The material may then be rinsed twice with 100-600 ml of sterile distilled water to remove residual detergent, which is removed from the container as described above. The processed tissue is then rinsed with 100% ethanol to remove residual free lipid. The majority of the solubilized lipid and ethanol may be removed from the container, as described above, and residual ethanol may be removed by evaporation, such as by gently warming the tissue and ethanol mixture. The collagen-rich material (processed acellular lipoaspirate) is then resuspended in saline and withdrawn into a syringe through an 18G needle. Fluid may need to be repeatedly passed through the needle in order to break up the collagen-rich material into particles small enough to pass through the 18G needle. The 18G needle may be replaced by a smaller gauge needle to allow further fragmentation of the processed-acellular lipoaspirate. In a preferred embodiment the size of the needle is serially reduced by replacing the larger needle with a smaller needle and repeating the procedure to the point where the processed acellular lipoaspirate flows freely through a needle of sufficiently small gauge to allow easy injection into sensitive locations such as the face. In other embodiments, the processed acellular lipoaspirate may be homogenized using other devices such as sonicators that help to break up the components of the processed acellular lipoaspirate.

Fragmentation of the collagen by use of shear force in a closed system may be achieved by repeatedly withdrawing and reinserting the material into and out of the container through a syringe with an 18G needle, for example, by inserting the needle of a syringe into the container containing the collagen-rich material, and withdrawing the material into the syringe, and expelling the material from the syringe while keeping the needle in the container. Once the material flows freely through the 18G needle the syringe is withdrawn and replaced with another bearing a smaller gauge needle and the process of withdrawing and re-inserting the material is repeated. This process is repeated with increasingly small needles until the material flows freely through the needle gauge intended for application.

In an exemplary embodiment, one solubilization liquid contains Triton X-100 detergent at concentrations from about 0.1% to about 5% and is incubated with tissue at from about 18° C. to about 38° C. for from about 20 minutes to about 60 minutes. These parameters will vary according to the amount of tissue to be digested and the degree of optional pre-washing with saline and/or distilled water, optimized by empirical studies, in order to validate that the system is effective at extracting the desired matrix material in an appropriate time frame. A particular preferred concentration, time and temperature is 2% Triton X-100 incubated for 45 minutes, at about 37° C. Alternate detergents such as Tween 20 and sodium dodecyl sulfate may also be applied.

Following solubilization of cellular components the collagen-rich material may be washed/rinsed to remove residual detergent and/or by-products of the solubilization process (e.g., solubilized cell components and newly-released free lipid). The collagen-rich material may also be washed with an organic solvent such as ethanol or acetone to remove residual free lipid. It could then be concentrated by centrifugation or other methods known to persons skilled in the art, as discussed above. These post-processing wash/concentration steps may be applied separately or simultaneously.

In addition to the foregoing, there are many post-wash methods that may be applied for further purifying the collagen-rich material. These include high salt washes, use of proteolytic enzymes that spare collagen, chemical modification of the material to modify the physical or physiologic properties of the material, or combinations thereof.

In one embodiment a combination of phenol, acetic acid, and water could be added to and mixed with the processed acellular lipoaspirate allowing for solubilization of non-collagen components contained in the processed acellular lipoaspirate (Davis and Mackle 1981).

In an alternate embodiment, the processed acellular lipoaspirate could be digested with proteolytic enzymes with limited ability to digest intact collagen (e.g., papain, pepsin, trypsin, etc.).

The final step of processing includes formulation of the collagen-rich material such that it is fragmented into particles small enough to be passed or administered through small gauge needles. This step is needed for applications in which the material will be implanted into body areas that are particularly sensitive such as the lips and nasio-labial folds. This can be achieved by mechanisms such as shear force (e.g., passage through small lumen apertures) or sonication. In a modified embodiment, which may be, for example, more expensive, only partially disposable and/or implemented in a non-closed system, the intact tissue fragments can be fragmented using rapidly spinning blades (chopping). Alternatively the collagen may be modified by esterification or treatment with dilute acid to increase solubility such that soluble collagen may be applied.

In certain embodiments, the collagen-rich material is administered directly into the patient. In other words, the material is administered to the patient without being removed from the system or exposed to the external environment of the system before being administered to the patient. Providing a closed system reduces the possibility of contamination of the material being administered to the patient. Thus, processing the adipose tissue in a closed system provides advantages over existing methods because the collagen-rich material is more likely to be sterile. In such an embodiment, the only time the material is exposed to the external environment, or removed from the system, is when the material is being withdrawn into an application device and being administered to the patient. In one embodiment, the application device can also be part of the closed system.

The material that has been concentrated, as described above, may be administered to a patient without further processing, or may be administered to a patient after being mixed with other tissues or cells. For example, the other tissue may comprise one or more units of lipoaspirate, collagen rich material or processed acellular lipoaspirate.

In other embodiments, at least a portion of the material is stored for later implantation/infusion. The processed acellular lipoaspirate may be divided into more than one aliquot or unit such that part of the material is retained for later application while part is applied immediately to the patient. Moderate to long-term storage of all or part of the material in a bank is also within the scope of this invention, as disclosed in U.S. patent application Ser. No. 10/242,094, entitled PRESERVATION OF NON EMBRYONIC CELLS FROM NON HEMATOPOIETIC TISSUES, filed Sep. 12, 2002, which claims the benefit of U.S. Provisional Patent Application 60/322,070 filed Sep. 14, 2001, which is commonly assigned, and the contents of which are expressly incorporated herein by reference.

At the end of processing, the material may be loaded into a delivery device, such as a syringe, for placement into the recipient by either subcutaneous, intramuscular, intraperitoneal, or periurethral techniques. In other words, cells may be placed into the patient by any means known to persons of ordinary skill in the art, for example, they may be injected into tissue (e.g., skeletal muscle), into the dermis (subcutaneous, facial applications), into tissue space (e.g., vocal fold), or into tissues (e.g., periurethral emplacement), or other location. Preferred embodiments include placement by needle or catheter, or by direct surgical implantation in association with additives such as a preformed matrix.

The material may be applied alone or in combination with cells, tissue, tissue fragments, demineralized bone, growth factors such as insulin or drugs such as members of the thiaglitazone family, biologically active or inert compounds, resorbable plastic scaffolds, or other additive intended to enhance the delivery, efficacy, tolerability, or function of the population. The material may also be modified by chemical means in such a way as to change, enhance, or supplement the function of the material for derivation of a cosmetic, structural, or therapeutic purpose. For example, esterification and cross linking may be applied to modify the solubility properties and/or the post-implantation stability of the material, as disclosed in U.S. Pat. No. 4,597,762.

In one aspect, the material could be formed into three dimensional structures for guided bone regeneration (Schwartzmann 2000) or to create a scaffold for three dimensional tissue engineering (Scherberich and Beretz 2000).

The material may also be administered to a patient for cosmetic purposes, such as by enhancing or improving physical features, including reducing wrinkles, enhancing organ mass, and the like.

A particular embodiment of components of tissue removal system is illustrated in FIGS. 1 and 2. A device 10 for separating collagen-rich material from adipose tissue is illustrated in FIG. 1. Device 10 includes a plurality of fluid containers 12A, 12B, 12C, and 12D connected to a tissue collection container 16 by way of tubing 14. Device 10 is illustrated as having four containers 12A, 12B, 12C, and 12D; however, more or fewer fluid containers may be provided depending on the number of reagents that are needed for solubilization and separation of the collagen-rich material from the extracted adipose tissue. In one embodiment, containers 12A, 12B, 12C, and 12D are flexible bags, and in other embodiments, containers 12A, 12B, 12C, and 12D are rigid containers. Containers 12A, 12B, 12C, and 12D include at least one aperture 13A, 13B, 13C, and 13D, respectively, for the addition and removal of a reagent. Containers 12A, 12B, 12C, and 12D having only one aperture will typically be filled with a reagent by delivering the reagent into containers 12A, 12B, 12C, and 12D through apertures 13A, 13B, 13C, and 13D, respectively.

After each of the containers 12A, 12B, 12C, and 12D receives a sufficient volume of a reagent, each of the apertures 13A, 13B, 13C, and 13D is connected to tubing 14 to permit the reagent to be delivered to tissue collection container 16. In the illustrated embodiment, containers 12A, 12B, 12C, and 12D have a plurality of apertures 13A, 15A, 13B, 15B, 13C, 15C, 13D, 15D, respectively. Apertures 13A, 13B, 13C, and 13D are illustrated as connected to tubing 14, and apertures 15A, 15B, 15C, and 15D are provided to permit addition of one or more reagents into containers 12A, 12B, 12C, and 12D. For example, each of the apertures 15A, 15B, 15C, and 15D may include a resealable membrane that permits a needle to be inserted therethrough to access the interior of a corresponding one of the containers 12A, 12B, 12C, and 12D, respectively. Needles may be used to add one or more reagents, as indicated above, or may be used to remove fluid from containers 12A, 12B, 12C, and 12D. Tubing 14 is preferably closed sterile tubing, or in other words, tubing 14 is not open to the external environment of device 10, and therefore provides a closed conduit from fluid containers 12A, 12B, 12C, and 12D to tissue collection container 16. Tubing 14 may be made of any material that can be provided sterilized, and preferably is a flexible tubing having a lumen size that can be controlled by one or more valves acting on the tubing, as discussed herein. In one embodiment, tubing and containers 12, 14 and 16 are made of polyethylene tubing. In one embodiment, tubing 14 would have a lumen diameter of greater than 2 mm, and preferably greater than 5 mm. These containers may have a volume of between approximately 200 ml to 1L. Such a system would have the ability to process up to 200 ml of adipose tissue and yield several grams of collagen-rich material.

Tissue collection container 16 includes one or more ports, such as ports 18 and 20 which are structured to provide access to the interior of tissue collection container 16. Tissue collection container 16 is a tissue collection container provided to collect and retain adipose tissue extracted from a patient. More particularly, tissue collection container 16 includes a port 18 that is dimensioned to be coupled to an aspiration device for aspirating adipose tissue. Port 18 is sufficiently large to permit relatively large units of adipose tissue to pass into tissue collection container 16. Tissue collection container 16 illustrated in FIG. 1 also includes a second port 20 provided for addition of one or more fluids to the interior of tissue collection container 16. In addition, or alternatively, second port 20 may also be used to remove material(s) contained within tissue collection container 16. As discussed herein, tissue collection container 16 preferably has a compressible body, such as body 17, which permits the contents in tissue collection container 16 to be agitated, as discussed herein. In one embodiment, tissue collection container 16 is a flexible bag made of a material that can be provided sterilized. Tissue collection container 16 also includes one or more apertures, such as aperture 19, to permit the reagents contained in containers 12 to be delivered to the interior of tissue collection container 16. In the illustrated embodiment, tissue collection container 16 includes one aperture 19 that is in communication with all four containers 12A, 12B, 12C, and 12D via tubing 14.

Device 10 also includes one or more components (flow control devices) to control the flow of reagents from containers 12A, 12B, 12C and 12D to tissue collection container 16. In the illustrated embodiment, device 10 includes a plurality of valves 22 disposed in-line between containers 12A, 12B, 12C and 12D and tissue collection container 16. In other words, one valve 22 is provided on each of the outflow lines of containers 12A, 12B, 12C and 12D. Thus, by actuating the valves 22 independently, a user can selectively control the addition of one or more reagents to tissue collection container 16. Valves 22 are preferably structured to prevent backflow of reagents into the other reagent containers when the valves of those containers are closed. For example, when a reagent within container 12A is being delivered to tissue collection container 16, the valve for container 12A is in an open position, and the valves for containers 12B, 12C and 12D may be in closed positions. Instead of, or in addition to, one or more clamps may be provided along tubing 14 to selectively control the flow of reagents into tissue collection container 16.

Thus, in the illustrated embodiment, device 10 is a device which is used to collect adipose tissue from a patient and which is not structured to be reused; or in other words, it is a single-use or disposable system. Accordingly, each of the components, such as containers 12A, 12B, 12C and 12D, tubing 14, and tissue collection container 16 can be pre-sterilized and disposed of after use with an individual patient.

In one specific embodiment of the invention, provided by way of example and not by way of limitation, one container 12A contains sterile distilled water, a second container 12B contains 2% Triton X-100 (Sigma, St. Louis, Mo.), a third container 12C contains 100% alcohol, such as ethanol, and a fourth container 12D contains saline, such as 0.9% NaCl in water. In this embodiment, each of the containers is dimensioned to contain approximately 1L of solution, or less.

In use, the tissue collection container 16 of device 10 is coupled to an aspiration device for liposuction. The extracted adipose tissue from a patient is delivered to tissue collection container 16, for example by way of port 18. As an optional step, the extracted tissue (which includes adipose tissue and non-adipose tissue) may be filtered and/or washed to remove free lipids and peripheral blood, as discussed above. For example, a filter may be provided in tissue collection container 16. One example of a filter includes a plurality of pores, of either the same or different sizes, but ranging in size from about 20 μm to 5 mm. In a preferred embodiment, the filter is a medical grade polyester mesh of around 200 μm thickness with a pore size of around 265 μm and around 47% open area. The cellular component of the intact adipose tissue that is contained within tissue collection container 16 is extracted with sterile water provided by container 12A. The water is selectively delivered to tissue collection container 16 by opening valve 22 on the tubing between container 12A and tissue collection container 16. The water and intact adipose tissue is mixed for a sufficient amount of time to extract the cellular component of the adipose tissue. This first step will remove the majority, preferably more than 80% of the cellular component of the adipose tissue. Tissue collection container 16 is then compressed to displace the soluble component, free lipid (from lysed fat cells), and water from the container. The water, lipid, and soluble component of the adipose tissue may then be removed from the container by withdrawing the water with the soluble component through a port, such as port 20. Or, the water, lipid, and soluble component may be removed from the container by expelling them back through tubing 14 into container 12A. Thus, tubing 14 provides a bidirectional fluid flow path between solubilizing liquid containers 12A, 12B, 12C, and 12D, and tissue collection container 16.

The precipitated material is then resuspended with the solution containing Triton X-100 from container 12B to provide additional solubilization of the cellular component of the adipose tissue. This detergent wash step will reduce the residual cellular component preferably by at least an additional 80%. The resulting supernatant is similarly removed, as described above. The free lipid component (typically, less than 1% of original lipid content of the adipose tissue) of the resulting precipitate is then solubilized by mixing the collagen-rich material with alcohol contained in container 12C. The insoluble component is then separated from the soluble component, as described above, and any residual alcohol is allowed to evaporate. Lastly, the precipitated collagen-rich material is resuspended in isotonic saline provided by container 12D. A needle, or other similar withdrawing device, is then inserted into tissue collection container 16, for example, by way of port 20, to remove the resuspended collagen-rich material. A typical collagen yield will be on the order of 2-10% of the dry weight of the original tissue depending largely on the Body Mass Index of the donor (a more overweight donor will have more fat per gram of tissue and therefore less collagen by a dilutive effect. The collagen-rich material may then be administered directly to a patient, or may be further processed to provide relatively finer suspensions of collagen-rich material. For example, the suspension of collagen rich material may be repeatedly passed through a series of different sized needles, or may be passed through filters of varying pore size to break down the size of particles within the suspension. Rhetorical question: in the above, which steps are part of a true closed system and which ones are not. Non-rhetorical question: is there any way to make this more of a closed system device? In an embodiment wherein all solutions are pre-loaded in containers 12A-D, the only non-closed parts in the system are the addition of the adipose tissue and the removal of the collagen-rich material.

In certain embodiments of the invention, one or more steps of processing the adipose tissue may be automated. For example, device 10 may be inserted into a processing system 40, such as that illustrated in FIG. 2. Processing system 40 is illustrated as including a base 44 and a holder assembly 42 having one or more holders 58. As presently embodied, holder assembly 42 has four holders 58, which are structured to hold containers 12A, 12B, 12C and 12D of device 10. Base 44 of processing system 40 is illustrated as including a front surface 46. Located on front surface 46 are a plurality of fittings 48 spaced apart from one another with a mixing element 50 disposed between fittings 48. Fittings 48 are structured to retain tissue collection container 16 of device 10. A pathway 52 is defined between fittings 48 such that mixing element 50 can move along pathway 52 to mix the contents within tissue collection container 16. In the illustrated embodiment, mixing element 50 is a roller bar that moves up and down along pathway 52 and compresses a region of tissue collection container 16 as it moves. This movement and compression of tissue collection container 16 causes agitation or mixing of the contents in tissue collection container 16. Tubing 14 of device 10 is placed in processing system 40 so that the flow of fluid through tubing 14 can be controlled by pump 54 and valves 56. Although the illustrated system is shown with four valves 56 and one pump 54, more or fewer valves or pumps may be provided, as discussed above. For example, four pumps 54 may be provided for each of the four containers 12A, 12B, 12C and 12D. Similarly, more or less fittings 48 may be provide to retain tissue collection container 16 on base 44.

As indicated above, in certain embodiments of the invention, the methods may be automated by providing one or more additional devices that can automatically perform the steps of the methods. In such embodiments, a processing device (e.g., microprocessor or personal computer) is a device to partially or completely automate the steps described above. Examples of steps amenable to such automation include, but are not limited to, controlling the ingress and egress of fluids and tissues along particular tubing paths by controlling pumps and valves of the system or processing device; detecting blockages with pressure sensors; mixing mechanisms, measuring the amount of tissue and/or fluid to be moved along a particular pathway using volumetric mechanisms; maintaining temperatures of the various components using heat control devices; washing the collagen-rich material, and integrating the process with timing and software mechanisms. In one embodiment, software can control the parameters of the process to allow production of a collagen-rich material prepared to specific operator-defined parameters. Thus, the automation device or devices improve the performance of the procedures, and provide automatic harvesting of adipose tissue and processing of the adipose tissue for administration to a patient.

Adipose tissue may be collected into the tissue collecting container while the container is in position within the device or prior to placement within the device.

In a further embodiment, software incorporated into the controller would prompt users through the steps necessary for proper insertion of tubing and other elements into the device. Software would also initiate automated testing to confirm correct insertion of tubing, absence of blockages, etc.

The general approach to processing in this device would use the same parameters as those described elsewhere in this disclosure for manual tissue processing.

Many other conformations of the staged mechanisms used for tissue processing will be apparent to one skilled in the art and the present description is included as one example only. For example, mixing of tissue and saline during washing and solubilization may occur by agitation as in the present example or by fluid recirculation. Tissue washing may be mediated by a moving bar mechanism such as shown here, or by rocking or other mechanism. Mechanisms for performance of such functions may be integrated within the device shown in FIG. 2 or may be incorporated in separate devices.

In a preferred embodiment of the invention, the tissue removal system and processing set is present in the vicinity of the patient receiving the treatment, such as the operating room or out-patient procedure room (effectively at the patient's bedside). This allows rapid, efficient tissue harvest and processing, reduces the opportunity for specimen handling/labeling errors, and thereby allows for performance of the entire process in the course of a single surgical procedure.

References:

Aragona, F., L. D'Urso, et al. (1998). “Immunologic aspects of bovine injectable collagen in humans. A review.” Eur Urol 33(2): 129-33.

Asken, S. (1990). “Microliposuction and autologous fat transplantation for aesthetic enhancement of the aging face.” J Dermatol Surg Oncol 16(10): 965-72.

Bartynski, J., M. S. Marion, et al. (1990). “Histopathologic evaluation of adipose autografts in a rabbit ear model.” Otolaryngol Head Neck Surg 102(4): 314-21.

Bergeon, M. T. (1967). “Collagen: a review.” J Okla State Med Assoc 60(6): 330-2.

Block, C. A., C. S. Cooper, et al. (2003). “Long-Term Efficacy of Periurethral Collagen Injection for the Treatment of Urinary Incontinence Secondary to Myelomeningocele.” J Urol 169(1): 327-329.

Boering, G. and A. J. Huffstadt (1967). “The use of derma-fat grafts in the face.” Br J Plast Surg 20(2): 172-8.

Boerner, C. F. (1988). “Allergic response to a porcine collagen corneal shield. Case report.” Arch Ophthalmol 106(2): 171.

Bulleid, N. J., D. C. John, et al. (2000). “Recombinant expression systems for the production of collagen.” Biochem Soc Trans 28(4): 350-3.

Burres, S. (2001). “Soft-tissue augmentation with Fascian.” Clin Plast Surg 28(1): 101-10.

Carpaneda, C. A. (1994). “Collagen alterations in adipose autografts.” Aesthetic Plast Surg 18(1): 11-5.

Coleman, S. R. (2001). “Structural fat grafts: the ideal filler?” Clin Plast Surg 28(1): 111-9.

Coleman, W. P., 3rd, N. Lawrence, et al. (1993). “Autologous collagen? Lipocytic dermal augmentation. A histopathologic study.” J Dermatol Surg Oncol 19(11): 1032-40.

Davis, P. F. and Z. M. Mackle (1981). “A simple procedure for the separation of insoluble collagen and elastin.” Anal Biochem 115(1): 11-7.

Ford, C. N., P. A. Staskowski, et al. (1995). “Autologous collagen vocal fold injection: a preliminary clinical study.” Laryngoscope 105(9 Pt 1): 944-8.

Groutz, A., J. G. Blaivas, et al. (2000). “Outcome results of transurethral collagen injection for female stress incontinence: assessment by urinary incontinence score.” J Urol 164(6): 2006-9.

Haab, F., P. E. Zimmern, et al. (1997). “Urinary stress incontinence due to intrinsic sphincteric deficiency: experience with fat and collagen periurethral injections.” J Urol 157(4): 1283-6.

Hamel, M., T. Shaarawy, et al. (2001). “Deep sclerectomy with collagen implant in patients with glaucoma and high myopia.” J Cataract Refract Surg 27(9): 1410-7.

Hsiung, M. W., P. Woo, et al. (2000). “Fat augmentation for glottic insufficiency.” Laryngoscope 110(6): 1026-33.

Kamer, F. M. and M. M. Churukian (1984). “Clinical use of injectable collagen. A three-year retrospective review.” Arch Otolaryngol 110(2): 93-8.

Klein, A. W. (2001). “Skin filling. Collagen and other injectables of the skin.” Dermatol Clin 19(3): 491-508, ix.

Koufman, J. A. (1991). “Lipoinjection for vocal cord paralysis.” Laryngoscope 101(12 Pt 1): 1385.

Latoni, J. D., D. M. Marshall, et al. (2000). “Overgrowth of fat autotransplanted for correction of localized steroid-induced atrophy.” Plast Reconstr Surg 106(7): 1566-9.

Lee, P. E., R. C. Kung, et al. (2001). “Periurethral autologous fat injection as treatment for female stress urinary incontinence: a randomized double-blind controlled trial.” J Urol 165(1): 153-8.

Liu, S. H., R. S. Yang, et al. (1995). “Collagen in tendon, ligament, and bone healing. A current review.” Clin Orthop(318): 265-78.

Miller, J. J. and J. C. Popp (2002). “Fat hypertrophy after autologous fat transfer.” Ophthal Plast Reconstr Surg 18(3): 228-31.

Mullins, R. J., C. Richards, et al. (1996). “Allergic reactions to oral, surgical and topical bovine collagen. Anaphylactic risk for surgeons.” Aust N Z J Ophthalmol 24(3): 257-60.

Myllyharju, J. (2000). “Recombinant collagen trimers from insect cells and yeast.” Methods Mol Biol 139: 39-48.

Nguyen, A., K. A. Pasyk, et al. (1990). “Comparative study of survival of autologous adipose tissue taken and transplanted by different techniques.” Plast Reconstr Surg 85(3): 378-86; discussion 387-9.

Ooi, K., M. P. Lacy, et al. (1991). “Salt-soluble collagen and elastin in the human aorta and pulmonary artery.” Exp Mol Pathol 55(1): 25-9.

Purna, S. K. and M. Babu (2000). “Collagen based dressings—a review.” Burns 26(1): 54-62.

Remacle, M., G. Lawson, et al. (1999). “Correcting vocal fold immobility by autologous collagen injection for voice rehabilitation. A short-term study.” Ann Otol Rhinol Laryngol 108(8): 788-93.

Scherberich, A. and A. Beretz (2000). “Culture of vascular cells in tridimensional (3-D) collagen: a methodological review.” Therapie 55(1): 35-41.

Schwartzmann, M. (2000). “Use of collagen membranes for guided bone regeneration: a review.” Implant Dent 9(1): 63-6.

Sclafani, A. P. and T. Romo, 3rd (2001). “Collagen, human collagen, and fat: the search for a three-dimensional soft tissue filler.” Facial Plast Surg 17(1): 79-85.

Sclafani, A. P., T. Romo, 3rd, et al. (2002). “Rejuvenation of the aging lip with an injectable acellular dermal graft (cymetra).” Arch Facial Plast Surg 4(4): 252-7.

Shore, J. W. (2000). “Injectable lyophilized particulate human fascia lata (Fascian) for lip, perioral, and glabellar enhancement.” Ophthal Plast Reconstr Surg 16(1): 23-7.

Silver, F. H. and G. Pins (1992). “Cell growth on collagen: a review of tissue engineering using scaffolds containing extracellular matrix.” J Long Term Eff Med Implants 2(1): 67-80.

Speranza, M. L. and G. Valentini (1986). “A simple procedure for the purification of neutral salt soluble type I collagen from skin.” Ital J Biochem 35(1): 42-8.

Takasaki, S., S. Fujiwara, et al. (1995). “Human type VI collagen: purification from human subcutaneous fat tissue and an immunohistochemical study of morphea and systemic sclerosis.” J Dermatol 22(7): 480-5.

Uitto, J. (1971). “Collagen biosynthesis in human skin. A review with emphasis on scleroderma.” Ann Clin Res 3(5): 250-8.

Yokoyama, T., N. Yoshimura, et al. (2001). “Persistence and survival of autologous muscle derived cells versus bovine collagen as potential treatment of stress urinary incontinence.” J Urol 165(1): 271-6.

Any feature or combination of features described herein are included within the scope of the present invention provided that the features included in any such combination are not mutually inconsistent as will be apparent from the context, this specification, and the knowledge of one of ordinary skill in the art. For purposes of summarizing the present invention, certain aspects, advantages and novel features of the present invention have been described herein. Of course, it is to be understood that not necessarily all such aspects, advantages or features will be embodied in any particular embodiment of the present invention. Additional advantages and aspects of the present invention are apparent in the following detailed description and claims.

The above-described embodiments have been provided by way of example, and the present invention is not limited to these examples. Multiple variations and modification to the disclosed embodiments will occur, to the extent not mutually exclusive, to those skilled in the art upon consideration of the foregoing description. Additionally, other combinations, omissions, substitutions and modifications will be apparent to the skilled artisan in view of the disclosure herein. Accordingly, the present invention is not intended to be limited by the disclosed embodiments, but is to be defined by reference to the appended claims.

A number of publications and patents have been cited herein. Each of the cited publications and patents are hereby incorporated by reference in their entireties.

What is claimed is:

1. A process for preparing a collagen-rich material for administration to a patient, comprising:
(a) removing adipose tissue from a patient, the adipose tissue including cellular, lipid and collagen components; and
(b) separating the collagen present in the adipose tissue from the cellular and lipid components of the adipose tissue to form a collagen-rich material suitable for immediate administration to the patient from which the adipose tissue was removed.

2. The process as set forth in claim 1, wherein the collagen-rich material comprises less than about 4 percent cells and cell fragments.

3. The process as set forth in claim 2, wherein the collagen-rich material is substantially free of cells and cell fragments.

4. The process as set forth in claim 3, wherein the collagen-rich material comprises less than about 0.1 percent cells and cell fragments.

5. The process as set forth in claim 1, wherein a percentage of reticular cellular components in the collagen-rich material, relative to a total amount of cellular components in the collagen-rich material, is less than or equal to a percentage of reticular cellular components in the removed adipose tissue, relative to a total amount of cellular components present in the removed adipose tissue.

6. The process as set forth in claim 1, wherein the collagen-rich material is neither dried nor preserved.

7. The process as set forth in claim 6, wherein the collagen-rich material is substantially free of cells and cell fragments.

8. The process as set forth in claim 1, and further comprising administrating the collagen-rich material into the patient from which the adipose tissue was removed.

9. The process as set forth in claim 8, wherein the collagen-rich material is substantially free of cells and cell fragments.

10. The process as set forth in claim 1, wherein (b) comprises one or more steps of solubilizing the cellular and lipid components of the adipose tissue with at least one solubilization liquid.

11. The process as set forth in claim 10, wherein (b) comprises mixing the adipose tissue with a solubilization liquid selected from the group consisting of sterile water, a solution having a detergent, and alcohol.

12. The process as set forth in claim 11, wherein (b) comprises separate steps of:
(i) mixing the adipose tissue with sterile water;
(ii) mixing the adipose tissue with a solution having a detergent; and
(iii) mixing the adipose tissue with alcohol.

13. The process as set forth in claim 1, and further comprising suspending the collagen-rich material in an isotonic solution before administering the collagen-rich material to the patient.

14. The process as set forth in claim 1, and further comprising one or more steps of dissociating the collagen-rich material to form a finer suspension of collagen-rich material than before dissociating.

15. A composition comprising a collagen-rich material prepared by the process of claim 1.

16. A composition comprising a collagen-rich material prepared by the process of claim 3.

17. A composition comprising a collagen-rich material prepared by the process of claim 10.

18. A composition comprising a collagen-rich material prepared by the process of claim 12.

19. A process for preparing a collagen-rich material for administration to a patient, comprising:
(a) removing adipose tissue from a patient, the adipose tissue having a protein component and a non-protein component; and
(b) solubilizing the non-protein component of the adipose tissue using at least one solubilizing liquid to obtain a collagen-rich material suitable for administration to a patient.

20. The process as set forth in claim 19, wherein substantially all of the non-protein component is solubilized.

21. The process as set forth in claim 19, wherein the liquid comprises at least one of sterile water, detergent and alcohol.

22. The process as set forth in claim 19, wherein the liquid is not an isotonic solution.

23. The process as set forth in claim 19, wherein collagen-rich material is substantially free of cells and cell fragments.

24. The process as set forth in claim 19, wherein a percentage of reticular cellular components in the collagen-rich material, relative to a total amount of cellular components in the collagen-rich material, is less than or equal to a percentage of reticular cellular components in the removed adipose tissue, relative to a total amount of cellular components present in the removed adipose tissue.

25. The process as set forth in claim 19, wherein (b) comprises separate steps of:
(i) mixing the adipose tissue with sterile water;
(ii) mixing the adipose tissue with a solution having a detergent; and
(iii) mixing the adipose tissue with alcohol.

26. The process as set forth in claim 25, and further comprising suspending the collagen-rich material in an isotonic solution before administering the collagen-rich material to the patient.

27. The process as set forth in claim 19, and further comprising one or more steps of dissociating the collagen-rich material to form a finer suspension of collagen-rich material than before dissociating.

28. A composition comprising a collagen-rich material prepared by the process of claim 19.

29. A composition comprising a collagen-rich material prepared by the process of claim 20.

30. A composition comprising a collagen-rich material prepared by the process of claim 24.

31. A device for processing adipose tissue obtained from a patient to obtain a collagen-rich material suitable for administration to a patient, comprising:
a tissue collection container structured to hold adipose tissue removed from a patient;
at least one solubilization liquid container; and
tubing connecting the at least one solubilization liquid container to the tissue collection container so that a solubilization liquid contained in the at least one solubilization liquid container can pass therefrom to the tissue collection container to be mixed with adipose tissue contained in the tissue collection container to form one or more solubilized components;
wherein the tissue collection container comprises a filter having a plurality of pores dimensioned to retain collagen rich material and to pass solubilized components of adipose tissue.

32. The device as set forth in claim 31, wherein pores of the plurality of pores have opening sizes between 20 microns and 5 mm.

33. The device as set forth in claim 31, wherein the tissue collection container comprises:
a flexible body;
an adipose tissue inlet port;
a solubilization liquid inlet port; and
a collagen-rich material outlet port.

34. The device as set forth in claim 31, wherein the at least one solubilization liquid container comprises a plurality of solubilization liquid containers.

35. The device as set forth in claim 34, wherein the at least one solubilization liquid container comprises four solubilization liquid containers, each container containing a different solubilization liquid.

36. The device as set forth in claim 35, wherein the device defines a closed system for processing the adipose tissue, the tubing comprising valves and the valves providing bidirectional flow between the tissue collection container and the four solubilization liquid containers.

37. The device as set forth in claim 34, wherein:
the device defines a closed system for processing the adipose tissue, and
the device further comprises a plurality of flow control devices positioned between the tissue collection container and the plurality of solubilization liquid containers to control the flow of fluid between the tissue collection container and the plurality of solubilization liquid containers.

38. The device as set forth in claim 37, wherein the flow control devices include at least one of a valve and a clamp.

39. The device as set forth in claim 31, wherein the components of the device are manufactured to be disposed of after a single use.

40. An automated system for processing adipose tissue obtained from a patient to obtain a collagen-rich material using the device of claim 31, comprising:
a base including at least one fitting to hold the tissue collection container, which is structured to hold adipose tissue removed from a patient;
a holder positioned with respect to the base to hold the at least one solubilization liquid container, which is coupled to the tissue collection container by the tubing;
an agitation element positioned to automatically agitate contents within the tissue collection container; and
a computer configured to selectively control the processing of adipose tissue within the tissue collection container to obtain a collagen-rich material suitable for administration to a patient.

41. The system as set forth in claim 40, and further comprising a pump disposed on the base in a position to pump a solubilization liquid from the at least one solubilization liquid container to the tissue collection container.

42. The system as set forth in claim 40, and further comprising at least one flow control device to control the flow of a solubilization liquid from the at least one solubilization liquid container.

43. The system as set forth in claim 42, wherein the at least one flow control device comprises a plurality of valves controlled by the computer.

44. The system as set forth in claim 40, wherein the agitation element comprises a roller bar moveable along the tissue collection container to agitate contents in the tissue collection container.
